(19) United States
(12) Patent Application Publication
(10) Pub. No.: US 2014/0207235 A1
(43) Pub. Date: Jul. 24, 2014

(54) EXPANDABLE ALLOGRAFT CAGE

(71) Applicant: WARSAW ORTHOPEDIC, INC., Warsaw, IN (US)

(72) Inventor: Susan J. Drapeau, Collierville, TN (US)

(73) Assignee: Warsaw Orthopedic, Inc., Warsaw, IN (US)

(21) Appl. No.: 13/748,227

(22) Filed: Jan. 23, 2013

(51) Int. Cl. A61F 2/44

(52) U.S. Cl. CPC A61F 2/442
USPC 623/17.16

(57) ABSTRACT

A spinal implant for insertion into and positioning in an intervertebral space is provided. The spinal implant includes an intervertebral cage having at least two cage ends and a spacer for insertion into the intervertebral cage between the at least two cage ends, the spacer configured to distract the intervertebral space to a desired height. The intervertebral cage and spacer comprise bone, and in some embodiments, allograft bone. A method for fusing two adjacent vertebrae utilizing the intervertebral allograft cage and spacer is also provided.

### FIELD OF INVENTION

The present application is directed to spinal implants, devices and methods for stabilizing vertebral members, and more particularly, to intervertebral implants, devices and methods of use in replacing, in whole or in part, an intervertebral disc, a vertebral member, or a combination of both to distract and/or stabilize the spine.

### BACKGROUND

The spine is divided into four regions comprising the cervical, thoracic, lumbar, and sacrococcygeal regions. The cervical region includes the top seven vertebral members identified as C1-C7. The thoracic region includes the next twelve vertebral members identified as T1-T12. The lumbar region includes five vertebral members L1-L5. The sacrococcygeal region includes nine fused vertebral members that form the sacrum and the coccyx. The vertebral members of the spine are aligned in a curved configuration that includes a cervical curve, thoracic curve, and lumbosacral curve. Intervertebral discs are positioned between the vertebral members and permit flexion, extension, lateral bending, and rotation.

Various conditions and ailments may lead to damage of the spine, intervertebral discs and/or the vertebral members. The damage may result from a variety of causes including, but not limited to, events such as trauma, a degenerative condition, a tumor, or infection. Damage to the intervertebral discs and vertebral members can lead to pain, neurological deficit, and/or loss of motion of the spinal elements.

Various procedures include replacing a section of or an entire intervertebral disc, a section of or an entire vertebral member, or both. One or more spinal implants may be inserted to replace damaged discs and/or vertebral members. The implants are configured to be inserted into an intervertebral space and contact against adjacent vertebral members. The implants are intended to reduce or eliminate the pain and neurological deficit, and increase the range of motion.

The curvature of the spine and general shapes of the vertebral members may make it difficult for the implants to adequately contact the adjacent vertebral members or to position the adjacent vertebral members in a desired orientation. There is a need for spinal implants or devices configurable to match the spinal anatomy for secure contact and/or desired orientation of the spinal implants or devices implanted into an intervertebral disc space.

In the spinal surgery field, surgical procedures are often performed to correct problems with displaced, damaged or degenerated intervertebral discs due to trauma, disease or aging. Bone graft materials are often used in spine fusion surgery. Current spinal fusion implants utilize grafts of either bone or artificial implants to fill the intervertebral disc space.

In particular, one method of treating a damaged disc is by immobilizing the area around the injured portion and fusing the immobilized portion by promoting bone growth between the immobilized spine portions. This often requires implantation of an intervertebral device to provide the desired spacing between adjacent vertebrae to maintain foraminal height and decompression. That is, an intervertebral implant comprising an interbody fusion device may be inserted into the intervertebral disc space of two neighboring vertebral bodies or into the space created by removal of damaged portions of the spine.

Formed implants are used to treat damaged vertebral disc. However, often these formed implants are available in limited sizes and shapes and provide little ability for customization. These formed implants also may not conform to cavities in the vertebral disc, which may make implantation of the implants at the target tissue site more challenging.

While generally effective, the use of bone grafts has some limitations. Autologous bone grafts, being obtained from the patient, require additional surgery and present increased risks associated with its harvesting, such as risk of infection, blood loss and compromised structural integrity at the donor site. Bone grafts using cortical bone remodel slowly because of their limited porosity. Traditional bone substitute materials and bone chips are more quickly remodeled but cannot immediately provide mechanical support. In addition, while bone substitute materials and bone chips can be used to fill oddly shaped bone defects. Indeed, the use of bone grafts is generally limited by the available shapes and sizes of grafts provided.

As to bone grafts, allograft bone is a reasonable bone graft substitute for autologous bone. It is readily available from cadavers and avoids the surgical complications and patient morbidity associated with harvesting autologous bone. Allograft bone is essentially a load-bearing matrix comprising cross-linked collagen, hydroxyapatite, and osteoinductive bone morphogenetic proteins. Human allograft tissue is widely used in orthopaedic surgery.

Xenograft bone is also another bone graft substitute where bone from one species is used in another species. The bone can be treated in order to reduce rejection by, for example, crosslinking, sintering, or use of supercritical carbon dioxide.

Indeed, allograft is a preferred material by surgeons for conducting interbody fusions because it will remodel over time into host bone within the fusion mass. However, though allograft tissue has certain advantages over the other treatments, allograft is typically available in only limited size ranges, thus making it difficult to provide implants, in particular, interbody implants in a preferred geometrical shape having an adjustable height. Accordingly, it would be desirable to construct an implant, particularly an interbody implant, to utilize better the benefits of allograft treatment. In some embodiments, the implant can be made from xenograft bone.

### SUMMARY

The present disclosure fills the foregoing need by providing a spinal implant for insertion into and positioning in an intervertebral space. The spinal implant comprises, consists essentially of, consists of an intervertebral cage having at least two cage ends and a spacer for insertion into the intervertebral cage between the at least two cage ends, the spacer configured to distract the intervertebral space to a desired height. The intervertebral cage and/or spacer comprise, consist essentially of, consist of bone, and in various embodiments, allograft bone or xenograft bone.

In some embodiments, the spacer comprises a spacer body, the spacer body having a spacer height, a spacer width, a spacer length and a spacer surface. In other embodiments, the spacer can be flat, cylindrical, conical, cuboid, shaped as a dowel, wedge, prism or pyramid. In some embodiments, the spacer can have various heights to allow for different expansion heights. In various embodiments, the cage ends and/or spacer have surface recesses or projections, for example, grooves or serrations positioned horizontally or at an angle from the horizontal.

According to one aspect, the intervertebral cage and/or spacer comprise demineralized allograft. The intervertebral cage and/or spacer comprise, consist essentially of, consist of demineralized bone in the core of their bodies and/or on the surface of their bodies.

According to other aspects, a method for fusing two adjacent vertebrae is provided, the method comprising the steps of providing an intervertebral cage having at least two ends and placing the intervertebral cage into an intervertebral space, providing a spacer having a spacer body, a spacer height, a spacer width, a spacer length and a spacer surface, implanting the spacer into the intervertebral cage, and orienting the spacer so that the intervertebral body distance is increased, wherein the intervertebral cage and the spacer are both formed of allograft bone.

According to yet another aspect, the intervertebral allograft cage and spacer are used in a surgical procedure comprising a posterior lumbar interbody fusion, transforaminal lumbar interbody fusion, an anterior lumbar interbody fusion or a direct lateral interbody fusion.

In other embodiments, a method of treating a spinal defect is provided, the method comprising providing a spinal cage having at least two ends opposite each other; preparing a disc space between adjacent vertebrae to receive the spinal cage; implanting the spinal cage into the prepared disc space; inserting a spacer between the two ends of the spinal cage; and orienting the spacer for interlocking with the spinal cage so that the intervertebral body distance is increased, wherein the spinal cage and the spacer are prepared of allograft.

While multiple embodiments are disclosed, still other embodiments of the present disclosure will become apparent to those skilled in the art from the following detailed description, which is to be read in connection with the accompanying drawing(s). As will be apparent, the disclosure is capable of modifications in various obvious aspects, all without departing from the spirit and scope of the present disclosure. Accordingly, the detailed description is to be regarded as illustrative in nature and not restrictive.

### BRIEF DESCRIPTION OF THE DRAWINGS

In part, other aspects, features, benefits and advantages of the embodiments will be apparent with regard to the following description, appended claims and accompanying drawings where:

FIG. 1 is a side view of an exemplary bone implant device according to one embodiment;

FIG. 2 is a side view of a spacer for a bone implant device according to another embodiment;

FIG. 3 is a side view of an exemplary interbody bone implant device containing a spacer according to an alternate embodiment; and

FIG. 4 is a side view of an exemplary interbody bone implant device containing a spacer according to yet another embodiment.

### DEFINITIONS

To aid in the understanding of the disclosure, the following non-limiting definitions are provided:

“Bioactive agent or bioactive compound,” as used herein, refers to a compound or entity that alters, inhibits, activates, or otherwise affects biological or chemical events. For example, bioactive agents may include, but are not limited to, osteogenic or chondrogenic proteins or peptides, anti-AIDS substances, anti-cancer substances, antibiotics, immunosuppressants, anti-viral substances, enzyme inhibitors, hormones, neurotoxins, opioids, hypnotics, anti-histamines, lubricants, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and antiadhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, angiogenic factors, anti-secretory factors, anticoagulants and/or antithrombotic agents, local anesthetics, ophthalmics, prostaglandins, anti-depressants, anti-psychotic substances, anti-emetics, and imaging agents. In certain embodiments, the bioactive agent is a drug. In some embodiments, the bioactive agent is a growth factor, cytokine, extracellular matrix molecule or a fragment or derivative thereof, for example, a cell attachment sequence such as RGD.

“Biocompatible,” as used herein, refers to materials that, upon administration in vivo, do not induce undesirable long-term effects.

“Bone,” as used herein, refers to bone that is cortical, cancellous or cortico-cancellous of autogenous, allogenic, xenogenic, or transgenic origin.

“Demineralized,” as used herein, refers to any material generated by removing mineral material from tissue, e.g., bone tissue. In certain embodiments, the demineralized compositions described herein include preparations containing less than 5% calcium and preferably less than 1% calcium by weight. Partially demineralized bone (e.g., preparations with greater than 5% calcium by weight but containing less than 100% of the original starting amount of calcium) is also considered within the scope of the disclosure. In some embodiments, demineralized bone has less than 95% of its original mineral content. Demineralized is intended to encompass such expressions as “substantially demineralized,” “partially demineralized,” and “fully demineralized.”

“Demineralized bone matrix” or “DBM” as used herein, refers to any material generated by removing mineral material from bone tissue. In some embodiments, the DBM compositions as used herein include preparations containing less than 5% calcium and preferably less than 1% calcium by weight. Partially demineralized bone (e.g., preparations with greater than 5% calcium by weight but containing less than 100% of the original starting amount of calcium) are also considered within the scope of the disclosure.

“Osteoconductive,” as used herein, refers to the ability of a non-osteoinductive substance to serve as a suitable template or substance along which bone may grow. In some embodiments, “osteoconduction” refers to the ability to stimulate the attachment, migration, and distribution of vascular and osteogenic cells within the graft material. The physical characteristics that affect the graft's osteoconductive activity include porosity, pore size, and three-dimensional architecture. In addition, direct biochemical interactions between matrix proteins and cell surface receptors play a major role in the host's response to the graft material.

“Osteogenic,” as used herein, refers to the ability of an agent, material, or implant to enhance or accelerate the growth of new bone tissue by one or more mechanisms such as osteogenesis, osteoconduction, and/or osteoinduction. In some embodiments, “osteogenic” refers to the ability of a graft material to produce bone independently. To have direct osteogenic activity, the graft must contain cellular components that directly induce bone formation. For example, a collagen matrix seeded with activated MSCs would have the potential to induce bone formation directly, without recruitment and activation of host MSC populations. Because many osteoconductive scaffolds also have the ability to bind and deliver bioactive molecules, their osteoinductive potential will be greatly enhanced.

“Osteoimplant,” as used herein, refers to any bone-derived implant prepared in accordance with the embodiments of this disclosure and therefore is intended to include expressions such as bone membrane, bone graft.

“Osteoinductive,” as used herein, refers to the quality of being able to recruit cells from the host that have the potential to stimulate new bone formation. Any material that can induce the formation of ectopic bone in the soft tissue of an animal is considered osteoinductive. In some embodiments, “osteoinduction” refers to the ability to stimulate the proliferation and differentiation of pluripotent mesenchymal stem cells (MSCs). In endochondral bone formation, stem cells differentiate into chondroblasts and chondrocytes, laying down a cartilaginous ECM, which subsequently calcifies and is remodeled into lamellar bone. In intramembranous bone formation, the stem cells differentiate directly into osteoblasts, which form bone through direct mechanisms. Osteoinduction can be stimulated by osteogenic growth factors, although some ECM proteins can also drive progenitor cells toward the osteogenic phenotype.

“Superficially demineralized,” as used herein, refers to bone-derived elements possessing at least about 90 weight percent of their original inorganic mineral content, the expression “partially demineralized” as used herein refers to bone-derived elements possessing from about 8 to about 90 weight percent of their original inorganic mineral content and the expression “fully demineralized” as used herein refers to bone containing less than 8% of its original mineral context.

The term “allograft” refers to a graft of tissue obtained from a donor of the same species as, but with a different genetic make-up from, the recipient, as a tissue transplant between two humans.

The term “autologous” refers to being derived or transferred from the same individual's body, such as for example an autologous bone marrow transplant.

The term “xenograft” refers to tissue or organs from an individual of one species transplanted into or grafted onto an organism of another species, genus, or family.

The term “morbidity” refers to the frequency of the appearance of complications following a surgical procedure or other treatment.

The term “patient” refers to a biological system to which a treatment can be administered. A biological system can include, for example, an individual cell, a set of cells (e.g., a cell culture), an organ, or a tissue. Additionally, the term “patient” can refer to animals, including, without limitation, humans.

The term “treating” or “treatment” of a disease refers to executing a protocol, which may include administering one or more drugs to a patient (human or otherwise), in an effort to alleviate signs or symptoms of the disease. Alleviation can occur prior to signs or symptoms of the disease appearing, as well as after their appearance. Thus, “treating” or “treatment” includes “preventing” or “prevention” of disease. In addition, “treating” or “treatment” does not require complete alleviation of signs or symptoms, does not require a cure, and specifically includes protocols, which have only a marginal effect on the patient.

### DETAILED DESCRIPTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Certain terminology, which may be used in the following description is for convenience only and is not limiting. For example, the words “right”, “left”, “top” and “bottom” designate directions in the drawings to which reference is made. The words, “anterior”, “posterior”, “superior”, “inferior”, “lateral” and related words and/or phrases designate preferred positions and orientations in the human body to which reference is made and are not meant to be limiting. The terminology includes the above-listed words, derivatives thereof and words of similar import.

Bone allograft is a preferred material by surgeons for conducting interbody fusions because it will remodel over time into host bone within the fusion mass. Polymer compositions also do not allow for direct bone attachment or bonding to further stabilize the implant and fusion mass. In addition, surgical procedures are increasingly moving towards minimally invasive surgical procedures in which smaller interbody cages can be inserted through smaller surgical incisions and expanded once placed in the disc space. The present disclosure overcomes the drawbacks of the prior art by providing various exemplary designs of bone implants comprising desirable allograft compositions that allow disc distraction and therefore stabilize the spine and increase height of adjacent vertebrae. The present disclosure also provides implants containing non-bone skeleton structures configured to include cavities including other materials, for example, allograft.

The present disclosure overcomes the drawbacks by providing various exemplary designs of bone implants comprising an intervertebral cage having at least two cage ends configured for receiving a spacer slidably insertable between the at least two cage ends configured to distract the intervertebral space to a desired height, wherein both the cage and the spacer comprise, consist essentially of or consist of bone. One exemplary configuration according to the present disclosure involves providing cortical allograft pieces that are mechanically interlocked together to form a single interbody implant. The cage comprises, consists essentially of or consists of a body of all allograft material which has at least two cage ends provided with grooves, recesses projections, serrations or combinations thereof configured to interlock with a spacer capable of distracting the cage into a disc space to a desired height. This design of the cage body and the spacer of an allograft allows for the advantageous properties of each to be fully realized.

In some embodiments, the grooves, recesses projections, serrations or combinations thereof of one or more portions of the cage allow corresponding grooves, recesses projections, serrations or combinations thereof of the spacer to fit within the cage and reduce or prevent movement of the spacer and allow for spine stabilization and the distance between vertebrae to be increased.

Various exemplary configurations according to the present disclosure involve providing cortical allograft constructs that mechanically interlock with an intervertebral cage and can slide into allograft cage to distract open the intervertebral disc space as they are inserted to form a single interbody implant. Advantageously, it is noted that an implant device may be provided in any configuration, size and shape, as per the requirements of the desired target site. Thus, an almost unlimited range of sizes and shapes of optimized bone implant devices may be provided. In one example, an implant device may be configured to be adapted for use as an interbody fusion device, e.g., in spinal fusion procedures. However, alternate configurations of the implant device are contemplated to suit the needs of a patient's bone graft target site.

In some embodiments, another advantage is that the expandable cage can be inserted through a small cannula into a disc space and once inserted in the disc space can be rotated to distract open the disc space to achieve the height required to remove nerve impingement or tissue encroachment. As a result, the expandable cages described in this disclosure can be inserted through smaller, minimally invasive surgical procedures that would allow physicians to perform with natural, tissue-based implants that can be easily incorporate into a patient's own bone. Moreover, these expandable cages can be designed for different approaches to the interbody space, such as for example, posterior lumbar interbody fusion (PLIF), transforaminal lumbar interbody fusion (TLIF), anterior lumbar interbody fusion (ALIF) and/or direct lateral interbody fusion (DLIF). The devices provided can be inserted through surgical techniques via open, mini-open, or minimal access spinal technologies or minimally invasive surgical techniques.

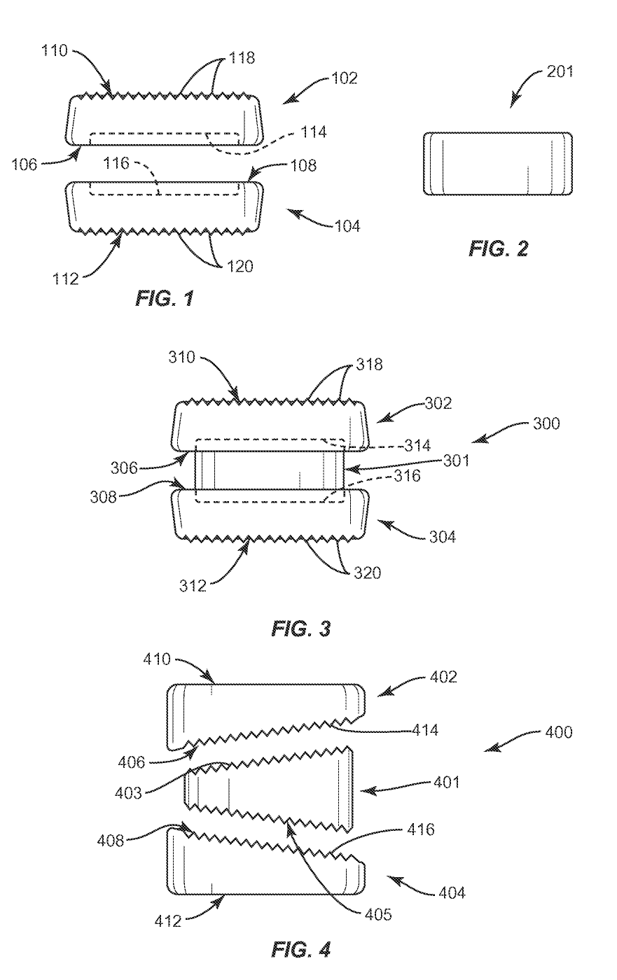

FIG. 1 depicts two cage ends 102, 104 of an interbody cage. Located between two intervertebral bodies, each cage end contains inner surfaces 106, 108 and outer surfaces, 110, 112. Inner surfaces 106, 108 comprise indentations or grooves 114, 116 configured to receive and interlock with a spacer (not shown) capable of distracting the cage into the disc space to the appropriate height. In some embodiments, the cage end creates a channel that allows the spacer to be inserted into. The outer surfaces 110, 112 can comprise serrations or teeth 118, 120 in order to grip more effectively into the intervertebral disc space. The outer surfaces 110, 112 are configured to contact different vertebrae and the spacer when inserted into the cage will increase the distance between vertebrae adjacent to the cage ends.

FIG. 2 is a side view of spacer 201 where all or a portion of the spacer 201 is configured to be inserted into the at least two cage ends and interlock to form spinal implant 300 as illustrated in FIG. 3. In various embodiments, spacer 201 can be flat or cylindrical. The height of spacer 201 can vary depending on the desired height and degree of stabilization needed.

FIG. 3 is a side view of the at least two cage ends 302, 304 comprising spacer 301. Each cage end 302, 304 contains inner surfaces, 306, 308 and outer surfaces, 310, 312. Inner surfaces 306, 308 comprise indentations or grooves 314, 316 configured to receive and interlock with a spacer 301, which spacer is capable of distracting cage ends 302, 304 into a selected disc space to the appropriate height. Outer surfaces 310, 312 of cage ends 302, 304 can comprise serrations or teeth 318, 320 in order to grip more effectively into the intervertebral disc space.

In another embodiment, in FIG. 4, cage ends 402, 404 of spinal implant 400 comprising spacer 401 are illustrated. Cage ends 402, 404 contain inner surfaces, 406, 408 and outer surfaces, 410, 412 Inner surfaces 406, 408 comprise serrations 414, 416 at an angle from the horizontal configured to receive and interlock with a spacer 401. In various embodiments, the angle from horizontal of inner surfaces 406, 408 can vary from about 0° to about 12°.

Conically shaped, spacer 401 also has serrations or teeth 403 on its outer surface 405 and is capable of distracting cage ends 402, 404 into a selected disc space to the appropriate height by rotating into serrations 414, 416 of cage ends 402, 404. In various embodiments, spacer 401 can have a height from about 1 mm to about 100 mm and a radius from about 1 mm to about 50 mm.

The term ‘cavity’ includes and encompasses voids, apertures, bores, depressions, holes, indentations, grooves, channels, notches or the like. In some embodiments, a plurality of cavities may be provided throughout one or more surfaces of and/or within the cage body and/or spacer thus enabling an additional plurality of allograft pieces to be retained in various locations of the expandable allograft cage.

Advantageously, the cage body and/or spacer may be formed via machining into any size or shape to accommodate the desired application and/or delivery conditions. The cage body and/or spacer may further be configured to include any desired features, such as cavities, projections, etc. in any desired location or orientation, as discussed further below.

The cage body including cage ends and/or spacer may also include a mechanism or features for reducing and/or preventing slippage or migration of the expandable implant device during insertion. For example, one or more surfaces of the cage body including the cage ends and/or spacer may include projections such as ridges or teeth as illustrated in FIGS. 1, 3 and 4 for increasing the friction between the implant device, spacer and the adjacent contacting surfaces of the vertebral bodies so to prevent movement of the implant device after introduction to a desired disc space.

In various embodiments, the delivery of the expandable implant device and its component pieces, for example the spacer, can be facilitated by using a lubricant to lubricate the implant device and its components prior to insertion into an intervertebral disc space. Suitable lubricants include without limitation mineral oils, bodily fluids, fat, saline or hydrogel coatings. Other useful lubricants include hyaluronic acid, hyaluronan, lubricin, polyethylene glycol and combinations thereof.

In some embodiments, the spinal allograft implant may comprise an allograft portion that is configured to be joined to another allograft portion. In this way, the interbody device can be joined before it is implanted at or near the target site. The composite interbody spinal implant can have mating surfaces comprising recesses and/or projections and reciprocating recesses and/or projections (e.g., joints) that allow the allograft implant to be assembled before implantation. Assembly can also include, for example, use of an adhesive material to join parts of the implant together and provide strong interlocking fit.

The adhesive material may comprise polymers having hydroxyl, carboxyl, and/or amine groups. In some embodiments, polymers having hydroxyl groups include synthetic polysaccharides, such as for example, cellulose derivatives, such as cellulose ethers (e.g., hydroxypropylcellulose). In some embodiments, the synthetic polymers having a carboxyl group, may comprise poly(acrylic acid), poly(methacrylic acid), poly(vinyl pyrrolidone acrylic acid-N-hydroxysuccinimide), and poly(vinyl pyrrolidone-acrylic acid-acrylic acid-N-hydroxysuccinimide) terpolymer. For example, poly(acrylic acid) with a molecular weight greater than 250,000 or 500,000 may exhibit particularly good adhesive performance. In some embodiments, the adhesive can be a polymer having a molecular weight of about 2,000 to about 5,000, or about 10,000 to about 20,000 or about 30,000 to about 40,000.

In some embodiments, the adhesive can comprise imido ester, p-nitrophenyl carbonate, N-hydroxysuccinimide ester, epoxide, isocyanate, acrylate, vinyl sulfone, orthopyridyl-disulfide, maleimide, aldehyde, iodoacetamide or a combination thereof. In some embodiments, the adhesive material can comprise at least one of fibrin, a cyanoacrylate (e.g., N-butyl-2-cyanoacrylate, 2-octyl-cyanoacrylate, etc.), a collagen-based component, a glutaraldehyde glue, a hydrogel, gelatin, an albumin solder, and/or a chitosan adhesives. In some embodiments, the hydrogel comprises acetoacetate esters crosslinked with amino groups or polyethers. In some embodiments, the adhesive material can comprise poly(hydroxylic) compounds derivatized with acetoacetate groups and/or polyamino compounds derivatized with acetoacetamide groups by themselves or the combination of these compounds crosslinked with an amino-functional crosslinking compounds.

The adhesive can be a solvent based adhesive, a polymer dispersion adhesive, a contact adhesive, a pressure sensitive adhesive, a reactive adhesive, such as for example multi-part adhesives, one part adhesives, heat curing adhesives, moisture curing adhesives, or a combination thereof or the like. The adhesive can be natural or synthetic or a combination thereof.

Contact adhesives are used in strong bonds with high shear-resistance. Pressure sensitive adhesives form a bond by the application of light pressure to bind the adhesive with the target tissue site, cannula and/or expandable member. In some embodiments, to have the device adhere to the target tissue site, pressure is applied in a direction substantially perpendicular to a surgical incision.

Multi-component adhesives harden by mixing two or more components, which chemically react. This reaction causes polymers to cross-link into acrylics, urethanes, and/or epoxies. There are several commercial combinations of multi-component adhesives in use in industry. Some of these combinations are: polyester resin-polyurethane resin; polyols-polyurethane resin, acrylic polymers-polyurethane resins or the like. The multi-component resins can be either solvent-based or solvent-less. In some embodiments, the solvents present in the adhesives are a medium for the polyester or the polyurethane resin. Then the solvent is dried during the curing process.

In some embodiments, the adhesive can be a one-part adhesive. One-part adhesives harden via a chemical reaction with an external energy source, such as radiation, heat, and moisture. Ultraviolet (UV) light curing adhesives, also known as light curing materials (LCM), have become popular within the manufacturing sector due to their rapid curing time and strong bond strength. Light curing adhesives are generally acrylic based. The adhesive can be a heat-curing adhesive, where when heat is applied (e.g., body heat), the components react and cross-link. This type of adhesive includes epoxies, urethanes, and/or polyimides. The adhesive can be a moisture curing adhesive that cures when it reacts with moisture present (e.g., bodily fluid) on the substrate surface or in the air. This type of adhesive includes cyanoacrylates or urethanes. The adhesive can have natural components, such as for example, vegetable matter, starch (dextrin), natural resins or from animals e.g. casein or animal glue. The adhesive can have synthetic components based on elastomers, thermoplastics, emulsions, and/or thermosets including epoxy, polyurethane, cyanoacrylate, or acrylic polymers.

In some embodiments, the interbody spinal bone implant may be joined together utilizing pins, rods, clips, or other fasteners to allow strong and easily coupling of component parts. In some embodiments, the allograft material is configured to provide the most contact to tissue surfaces (e.g., the allograft material can be on the perimeter of the device, while the polymer material is situated in the interior of the device).

In addition to the cage body and/or spacer being enabled to be provided in various configurations, shapes and sizes, the cage body may include any number of cavities in different arrangements, locations, sizes and shapes. For example, the arrangement and location of cavities may be determined based on application of the expandable implant device.

In some embodiments, the surfaces of the cage body and/or spacer include at least one cavity or a plurality of cavities (not shown). Each cavity may be provided in any of a variety of shapes in addition to the generally rectangular shapes including but not limited to generally circular, oblong, curved, triangular and other polygonal or non-polygonal shapes. The same or different types of cavity shapes and sizes may be provided in each cage body and/or spacer. Each cavity may be formed to pass entirely through the cage body and/or spacer for promoting fusion between the upper and lower vertebral bodies. Alternately, cavities may be formed to partially pass through the cage body and/or spacer, or may be formed only on one or more surfaces thereof.

According to some embodiments, fusion may be facilitated or augmented by introducing or positioning various osteoinductive materials within the cavities in the expandable implant device. Such osteoinductive materials may be introduced before, during, or after insertion of the exemplary implant device, and may include (but are not necessarily limited to) autologous bone harvested from the patient receiving the implant device, bone allograft, bone xenograft, bone morphogenic protein, and/or bio-resorbable compositions.

A substance such as a biocompatible material may be inserted and retained within the cavities of the allograft cage body and/or spacer. The biocompatible material may comprise an osteoinductive material.

In one embodiment, the osteoinductive material comprises allograft tissue. Non-limiting examples of a bone graft material include demineralized bone matrix, or a bone composite. While allograft bone is a desirable alternative to autograft, it must be rigorously processed and terminally sterilized prior to implantation to remove the risk of disease transmission or an immunological response. This processing removes the osteogenic and osteoinductive properties of the allograft, leaving only an osteoconductive scaffold.

In one embodiment, to improve the osteoinductive properties, it is desirable to use demineralized bone matrix (DBM) as the osteoinductive material, due to its superior biological properties relative to undemineralised allograft bone, since the removal of minerals increases the osteoinductivity of the graft. Currently, there are a range of DBM products in clinical use.

Demineralized bone matrix (DBM) is demineralized allograft bone with osteoinductive activity. DBM is prepared by acid extraction of allograft bone, resulting in loss of most of the mineralized component but retention of collagen and noncollagenous proteins, including growth factors. DBM does not contain osteoprogenitor cells, but the efficacy of a demineralized bone matrix as a bone-graft substitute or extender may be influenced by a number of factors, including the sterilization process, the carrier, the total amount of bone morphogenetic protein (BMP) present, and the ratios of the different BMPs present. DBM includes demineralized pieces of cortical bone to expose the osteoinductive proteins contained in the matrix. These activated demineralized bone particles are usually added to a substrate or carrier (e.g. glycerol or a polymer). DBM is mostly an osteoinductive product, but lacks enough induction to be used on its own in challenging healing environments such as posterolateral spine fusion.

According to some embodiments of the disclosure, the demineralized bone matrix may comprise demineralized bone matrix fibers and/or demineralized bone matrix chips. In some embodiments, the demineralized bone matrix may comprise demineralized bone matrix fibers and demineralized bone matrix chips in a 30:70 ratio.

The demineralized allograft bone material may be further modified such that the original chemical forces naturally present have been altered to attract and bind growth factors, other proteins and cells affecting osteogenesis, osteoconduction and osteoinduction. For example, a demineralized allograft bone material may be modified to provide an ionic gradient to produce a modified demineralized allograft bone material, such that implanting the modified demineralized allograft bone material results in enhanced ingrowth of host bone.

In one embodiment an ionic force change agent may be applied to modify the demineralized allograft bone material. The demineralized allograft bone material may comprise, e.g., a demineralized bone matrix (DBM) comprising fibers, particles and any combination of thereof According to another embodiment, a bone graft structure may be used which comprises a composite bone, which includes a bone powder, a polymer and a demineralized bone.

The ionic force change agent may be applied to the entire demineralized allograft bone material or to selected portions/surfaces thereof.

The ionic force change agent may be a binding agent, which modifies the demineralized allograft bone material or bone graft structure to bind molecules, such as, for example, growth factors, or cells, such as, for example, cultured cells, or a combination of molecules and cells. In the practice of the disclosure the growth factors include but are not limited to BMP-2, rhBMP-2, BMP-4, rhBMP-4, BMP-6, rhBMP-6, BMP-7(OP-1), rhBMP-7, GDF-5, LIM mineralization protein, platelet derived growth factor (PDGF), transforming growth factor-β (TGF-β), insulin-related growth factor-I (IGF-I), insulin-related growth factor-II (IGF-II), fibroblast growth factor (FGF), beta-2-microglobulin (BDGF II), and rhGDF-5. A person of ordinary skill in the art will appreciate that the disclosure is not limited to growth factors only. Other molecules can also be employed in the disclosure. For example, tartrate-resistant acid phosphatase, which is not a growth factor, may also be used in the disclosure.

If a cell culture is employed, the cells include but are not limited to mesenchymal stems cells, pluripotent stem cells, osteoprogenitor cells, osteoblasts, osteoclasts, and any bone marrow-derived cell lines.

In some embodiments, the ionic force change agent comprises at least one of enzymes, enzyme mixtures, pressure (e.g., isostatic pressure), chemicals, heat, sheer force, oxygen plasma, or a combination thereof. For example, the ionic force change agent may comprise an enzyme such as collagenase or pepsin, which can be administered for a sufficient period of time to partially digest at least a portion of the demineralized allograft bone material. Subsequently, the enzyme may be deactivated and/or removed.

Any enzyme or enzyme mixture may be contemplated, and treatment time durations may be altered in accordance with the enzyme(s) used. Some suitable enzymes that may degrade the DBM material include, but are not limited to, cysteine proteinases, matrix metalloproteinases, enzymes such as amylases, proteases, lipases, pectinases, cellulases, hemicellulases, pentosanases, xylanases, phytases or combinations thereof. Exemplary enzymes suitable to partially degrade and modify the DBM material, include but are not limited to, cathepsin L, cathepsin K, cathepsin B, collagenase, pepsin, plasminogen, elastase, stromelysin, plasminogen activators, or a combination thereof.

In some embodiments, the DBM material can be subjected to pressure to modify it. The simplest pressing technique is to apply pressure to the unconstrained DBM material. Examples include pressing the DBM material using a mortar and pestle, applying a rolling/pressing motion such as is generated by a rolling pin, or pressing the bone pieces between flat or curved plates. These flattening pressures cause the DBM material fibers to remain intact.

Another pressing technique involves mechanically pressing demineralized bone material, which can be constrained within a sealed chamber having a hole (or a small number of holes) in its floor or bottom plate. The separated fibers extrude through the holes with the hole diameter limiting the maximum diameter of the extruded fibers. This constrained technique results in fibers that are largely intact (as far as length is concerned).

In a combined unconstrained/constrained pressing technique that results in longer fibers by minimizing fiber breakage, the demineralized bone is first pressed into an initially separated mass of fibers while in the unconstrained condition and thereafter these fibers are constrained within the sealed chamber where pressing is continued.

In general, pressing of demineralized bone to provide demineralized bone particles can be carried out at from about 1,000 to about 40,000 psi, and preferably at from about 5,000 to about 20,000 psi.

Subsequent to the addition of the ionic force change agent, the practitioner may optionally administer an appropriate molecule or cell culture. Generally, the molecule or cell culture is applied within minutes, for example from about 1 to about 120 minutes before implantation into the patient.

One class of molecules suitable for one embodiment of the disclosure is growth factors. Growth factors suitable for use in the practice of the disclosure include but are not limited to bone morphogenic proteins, for example, BMP-2, rhBMP-2, BMP-4, rhBMP-4, BMP-6, rhBMP-6, BMP-7 (OP-1), rhBMP-7, GDF-5, and rhGDF-5. Bone morphogenic proteins have been shown to be excellent at growing bone and there are several products being tested. rhBMP-2 can also be used on a carrier for acute, open fractures of the tibial shaft. BMP-7 (OP-1) also enhances bone growth in a posterolateral fusion procedure.

Additionally, suitable growth factors include, without limitation, LIM mineralization protein, platelet derived growth factor (PDGF), transforming growth factor β (TGF-β), insulin-related growth factor-I (IGF-I), insulin-related growth factor-II (IGF-II), fibroblast growth factor (FGF), and beta-2-microglobulin (BDGF II).

Further, molecules, which do not have growth factor properties, may also be suitable for this disclosure. An example of such molecules is tartrate-resistant acid phosphatase.

In one embodiment, the demineralized allograft bone material is treated with a negatively-charged ionic force change agent to produce a negatively-charged demineralized allograft bone material. The negatively-charged demineralized allograft bone material attracts a positively charged molecule having a pI from about 8 to about 10. Examples of positively charged molecules having a pI from about 8 to about 10 include but are not limited to, rhBMP-2 and rhBMP-6.

In another embodiment, the demineralized allograft bone material is treated with a positively-charged ionic force change agent such that the positively-charged demineralized allograft bone material attracts a molecule with a slightly negative charge, for example a charge of pI about 5 to about 7. Examples of molecules having a slightly negative charge include rhBMP-4.

In yet another embodiment, the demineralized allograft bone material is treated with a positively-charged ionic force change agent to produce a positively-charged demineralized allograft bone material such that cells, in particular cell cultures having a negative surface charge bind to the positively-charged demineralized allograft bone material. Examples of cells which are suitable for use in the practice of the disclosure include but are not limited to mesenchymal stems cells, pluripotent stem cells, embryonic stem cells, osteoprogenitor cells and osteoblasts.

The mechanisms by which a demineralized allograft bone material may acquire ionic forces include but are not limited to ionization, ion adsorption and ion dissolution.

In one embodiment, the implant is modified to give it the selected charge by a one-to-one substitution of the calcium ion with lithium, sodium, potassium or cesium of hydroxyapatite.

In yet another aspect, treatments with gradient-affecting elements, such as elements present in hydroxyapatite, and human proteins are employed. Suitable gradient-affecting proteins are those present in the organic phase of human bone tissue. The gradient-affecting proteins derive molecule or cell attraction without the potential damaging effects on the implants, as may be the case with other chemical treatments. Usually this is accomplished through surface treatments such as, for example, plasma treatment to apply an electrostatic charge on bone.

The term “plasma” in this context is an ionized gas containing excited species such as ions, radicals, electrons and photons. The term “plasma treatment” refers to a protocol in which a surface is modified using a plasma generated from process gases including, but not limited to, O₂, He, N₂, Ar and N₂O. To excite the plasma, energy is applied to the system through electrodes. This power may be alternating current (AC), direct current (DC), radiofrequency (RF), or microwave frequency (MW). The plasma may be generated in a vacuum or at atmospheric pressure. The plasma can also be used to deposit polymeric, ceramic or metallic thin films onto surfaces. Plasma treatment is an effective method to uniformly alter the surface properties of substrates having different or unique size, shape and geometry including but not limited to bone and bone composite materials.

Having been deposited in the disc space, an implant device of the present disclosure effects spinal fusion over time as the natural healing process integrates and binds the implant within the intervertebral space by allowing a boney bridge to form through the implant and between the adjacent vertebral bodies.

In some embodiments, an implant device of the present disclosure may be used to deliver substances such as surface demineralized bone chips, optionally of a predetermined particle size, demineralized bone fibers, optionally pressed, and/or allograft.

For embodiments where the substance is biologic, the substance may be autogenic, allogenic, xenogenic, or transgenic. However, it is contemplated that other suitable materials may be positioned in the implant device such as, for example, protein, nucleic acid, carbohydrate, lipids, collagen, allograft bone, autograft bone, cartilage stimulating substances, allograft cartilage, TCP, hydroxyapatite, calcium sulfate, polymer, nanofibrous polymers, growth factors, carriers for growth factors, growth factor extracts of tissues, demineralized bone matrix, dentine, bone marrow aspirate, bone marrow aspirate combined with various osteoinductive or osteoconductive carriers, concentrates of lipid derived or marrow derived adult stem cells, umbilical cord derived stem cells, adult or embryonic stem cells combined with various osteoinductive or osteoconductive carriers, transfected cell lines, bone forming cells derived from periosteum, combinations of bone stimulating and cartilage stimulating materials, committed or partially committed cells from the osteogenic or chondrogenic lineage, or combinations of any of the above. In some embodiments, the substance may be pressed before placement in the implant device. A substance provided within the implant device may be homogenous, or generally a single substance, or may be heterogeneous, or a mixture of substances.

In some embodiments the substance delivered by the implant device may include or comprise an additive such as an angiogenesis promoting material or a bioactive agent. It will be appreciated that the amount of additive used may vary depending upon the type of additive, the specific activity of the particular additive preparation employed, and the intended use of the composition. The desired amount is readily determinable by one skilled in the art. Angiogenesis may be an important contributing factor for the replacement of new bone and cartilage tissues. In certain embodiments, angiogenesis is promoted so that blood vessels are formed at an implant site to allow efficient transport of oxygen and other nutrients and growth factors to the developing bone or cartilage tissue. Thus, angiogenesis promoting factors may be added to the substance to increase angiogenesis. For example, class 3 semaphorins, for example, SEMA3, controls vascular morphogenesis by inhibiting integrin function in the vascular system, and may be included in the recovered hydroxyapatite.

In accordance with some embodiments, the substance may be supplemented, further treated, or chemically modified with one or more bioactive agents or bioactive compounds. Bioactive agent or bioactive compound, as used herein, refers to a compound or entity that alters, inhibits, activates, or otherwise affects biological or chemical events. For example, bioactive agents may include, but are not limited to, osteogenic or chondrogenic proteins or peptides; demineralized bone powder; collagen, insoluble collagen derivatives, etc., and soluble solids and/or liquids dissolved therein; anti-AIDS substances; anti-cancer substances; antimicrobials and/or antibiotics such as erythromycin, bacitracin, neomycin, penicillin, polymycin B, tetracyclines, biomycin, chloromycetin, and streptomycins, cefazolin, ampicillin, azactam, tobramycin, clindamycin and gentamycin, etc.; immunosuppressants; anti-viral substances such as substances effective against hepatitis; enzyme inhibitors; hormones; neurotoxins; opioids; hypnotics; anti-histamines; lubricants; tranquilizers; anti-convulsants; muscle relaxants and anti-Parkinson substances; anti-spasmodics and muscle contractants including channel blockers; miotics and anti-cholinergics; anti-glaucoma compounds; anti-parasite and/or anti-protozoal compounds; modulators of cell-extracellular matrix interactions including cell growth inhibitors and antiadhesion molecules; vasodilating agents; inhibitors of DNA, RNA, or protein synthesis; anti-hypertensives; analgesics; anti-pyretics; steroidal and non-steroidal anti-inflammatory agents; anti-angiogenic factors; angiogenic factors and polymeric carriers containing such factors; anti-secretory factors; anticoagulants and/or antithrombotic agents; local anesthetics; ophthalmics; prostaglandins; anti-depressants; anti-psychotic substances; anti-emetics; imaging agents; biocidal/biostatic sugars such as dextran, glucose, etc.; amino acids; peptides; vitamins; inorganic elements; co-factors for protein synthesis; endocrine tissue or tissue fragments; synthesizers; enzymes such as alkaline phosphatase, collagenase, peptidases, oxidases, etc.; polymer cell scaffolds with parenchymal cells; collagen lattices; antigenic agents; cytoskeletal agents; cartilage fragments; living cells such as chondrocytes, bone marrow cells, mesenchymal stem cells; natural extracts; genetically engineered living cells or otherwise modified living cells; expanded or cultured cells; DNA delivered by plasmid, viral vectors, or other means; tissue transplants; autogenous tissues such as blood, serum, soft tissue, bone marrow, etc.; bioadhesives; bone morphogenic proteins (BMPs); osteoinductive factor (IFO); fibronectin (FN); endothelial cell growth factor (ECGF); vascular endothelial growth factor (VEGF); cementum attachment extracts (CAE); ketanserin; human growth hormone (HGH); animal growth hormones; epidermal growth factor (EGF); interleukins, e.g., interleukin-1 (IL-1), interleukin-2 (IL-2); human alpha thrombin; transforming growth factor (TGF-β); insulin-like growth factors (IGF-1, IGF-2); parathyroid hormone (PTH); platelet derived growth factors (PDGF); fibroblast growth factors (FGF, BFGF, etc.); periodontal ligament chemotactic factor (PDLGF); enamel matrix proteins; growth and differentiation factors (GDF); hedgehog family of proteins; protein receptor molecules; small peptides derived from growth factors above; bone promoters; cytokines; somatotropin; bone digesters; antitumor agents; cellular attractants and attachment agents; immuno-suppressants; permeation enhancers, e.g., fatty acid esters such as laureate, myristate and stearate monoesters of polyethylene glycol, enamine derivatives, alpha-keto aldehydes, etc.; and nucleic acids.

In certain embodiments, the bioactive agent may be a drug. In some embodiments, the bioactive agent may be a growth factor, cytokine, extracellular matrix molecule, or a fragment or derivative thereof, for example, a protein or peptide sequence such as RGD.

In one embodiment of an implant device comprising at least one cavity, it may be contemplated that any combination or mixture of same or different substances may be placed and retained therein, and further, different substances may be placed within the same or different cavities.

### Sterilization

A medical implant device according to the present disclosure including its contents may be sterilizable. In various embodiments, one or more components of the implant device and/or its contents are sterilized by radiation in a terminal sterilization step in the final packaging. Terminal sterilization of a product provides greater assurance of sterility than from processes such as an aseptic process, which require individual product components to be sterilized separately and the final package assembled in a sterile environment.

In various embodiments, gamma radiation is used in the terminal sterilization step, which involves utilizing ionizing energy from gamma rays that penetrates deeply in the device. Gamma rays are highly effective in killing microorganisms, they leave no residues nor have sufficient energy to impart radioactivity to the device. Gamma rays can be employed when the device is in the package and gamma sterilization does not require high pressures or vacuum conditions, thus, package seals and other components are not stressed. In addition, gamma radiation eliminates the need for permeable packaging materials.

In various embodiments, electron beam (e-beam) radiation may be used to sterilize one or more components of the device. E-beam radiation comprises a form of ionizing energy, which is generally characterized by low penetration and high-dose rates. E-beam irradiation is similar to gamma processing in that it alters various chemical and molecular bonds on contact, including the reproductive cells of microorganisms. Beams produced for e-beam sterilization are concentrated, highly-charged streams of electrons generated by the acceleration and conversion of electricity. E-beam sterilization may be used, for example, when the medical device has gel components.

Other methods may also be used to sterilize the device and/or one or more components of the device and/or contents, including, but not limited to, gas sterilization, such as, for example, with ethylene oxide or steam sterilization.

### Method of Use

An intervertebral implant device according to the present disclosure delivers the substance or substances in vivo. Active delivery of the substance may include the cleavage of physical and/or chemical interactions of substance from covering with the presence of body fluids, extracellular matrix molecules, enzymes or cells. Further, it may comprise formation change of substances (growth factors, proteins, polypeptides) by body fluids, extracellular matrix molecules, enzymes or cells.

The body of the implant device is loaded with the substance for placement in vivo. The body may be pre-loaded, thus loaded at manufacture, or may be loaded in the operating room or at the surgical site. Preloading may be done with any of the substances previously discussed including, for example, allograft such as DBM, synthetic calcium phosphates, synthetic calcium sulfates, enhanced DBM, collagen, carrier for stem cells, and expanded cells (stem cells or transgenic cells). Loading in the operating room or at the surgical site may be done with any of these materials and further with autograft and/or bone marrow aspirate.

Any suitable method may be used for loading a substance in the implant device in the operating room or at the surgical site. For example, the substance may be spooned into the cavity(ies) of the implant device, the substance may be placed in the implant device using forceps, the substance may be loaded into the implant device using a syringe (with or without a needle), or the substance may be inserted into the implant device in any other suitable manner. Specific embodiments for loading at the surgical site include for example, vertebroplasty or interbody space filler.

For placement, the substance or substances may be provided in the implant device and the implant device placed in vivo. In one embodiment, the implant device is placed in vivo by placing the implant device in a catheter or tubular inserter and delivering the implant device with the catheter or tubular inserter. The implant device, with a substance provided therein, may be steerable such that it can be used with flexible introducer instruments for, for example, minimally invasive spinal procedures. For example, the implant device may be introduced down a tubular retractor, cannula or scope, during PLIF, TLIF, ALIF, DLIF or other procedures. In other embodiments, the implant device (with or without substance loaded) may be placed in a cage, for example, for interbody fusion.

Attachment mechanisms provided on the implant device may be used to couple the device to a site in vivo.

An implant device according to the present disclosure may be configured for use in any suitable application. In some embodiments, the implant device may be used in healing vertebral compression fractures, interbody fusion, minimally invasive procedures, posterolateral fusion, correction of adult or pediatric scoliosis and others. (please verify) The implant device may be used in a minimally invasive procedure via placement through a small incision, via delivery through a tube, or other. The size and shape of the device may advantageously be designed in accordance with restrictions on delivery conditions.

An exemplary application for using an implant device as disclosed is fusion of the spine. In clinical use, the implant device and delivered substance may be used to bridge the gap between the transverse processes of adjacent or sequential vertebral bodies. The implant device may be used to bridge two or more spinal motion segments. The implant device surrounds the substance to be implanted, and contains the substance to provide a focus for healing activity in the body.

In other applications, the implant device may be applied to transverse processes or spinous processes of vertebrae.

Generally, the implant device may be applied to a pre-existing defect, to a created channel, or to a modified defect. Thus, for example, a channel may be formed in a bone, or a pre-existing defect may be cut to form a channel, for receipt of the implant device. The implant device may be configured to match the channel or defect. In some embodiments, the configuration of the implant device may be chosen to match the channel. In other embodiments, the channel may be created, or the defect expanded or altered, to reflect a configuration of the implant device. The implant device may be placed in the defect or channel and, optionally, coupled using attachment mechanisms.

At the time just prior to when the implant device is to be placed in a defect site, optional materials, e.g., autograft bone marrow aspirate, autograft bone, preparations of selected autograft cells, autograft cells containing genes encoding bone promoting action, etc., can be combined with the implant device and/or with a substance provided in the implant device. The implant device can be implanted at the bone repair site, if desired, using any suitable affixation means, e.g., sutures, staples, bioadhesives, screws, pins, rivets, other fasteners and the like or it may be retained in place by the closing of the soft tissues around it.

Although the disclosure has been described with reference to some embodiments, persons skilled in the art will recognize that changes may be made in form and detail without departing from the spirit and scope of the disclosure.

1. A spinal implant for positioning between adjacent vertebrae, the spinal implant comprising: an intervertebral cage having at least two cage ends, each cage end configured to contact adjacent vertebrae, a spacer configured for insertion into the intervertebral cage between the at least two cage ends so as to distract the adjacent vertebrae to a desired height, the spacer comprising a length that is smaller than a length of the at least two cage ends, wherein the intervertebral cage and spacer are made entirely of bone and the bone is positively charged or negatively charged.

2. A spinal implant according to claim 1, wherein the bone is negatively charged with a negatively-charged ionic force change agent or is positively charged with a positively-charged ionic force change agent.

3. A spinal implant according to claim 1, wherein the at least two cage ends and the spacer are made entirely of allograft bone or xenograft bone.

4. A spinal implant according to claim 1, wherein the spacer comprises a body that is a preformed osteoconductive insert.

5. A spinal implant according claim 1, wherein the intervertebral cage and/or spacer comprise demineralized allograft.

6. A spinal implant according to claim 5, wherein the intervertebral cage and/or spacer have a portion comprising demineralized bone.

7. A spinal implant according to claim 1, wherein the spacer comprises a body that has a dowel, wedge, cylinder, prism, cuboid, cone or pyramid shape.

8. A spinal implant according to claim 2, wherein the intervertebral cage and/or spacer comprise grooves, recesses, projections, serrations or combinations thereof.

9. A spinal implant according to claim 8, wherein the serrations have shapes comprising triangular, pyramid, cone, spike, keel shapes or combinations thereof.

10. A spinal implant according to claim 1, wherein the spacer is configured to impart a substantially vertical expansion to the spinal implant.

11. A spinal implant according to claim 2, wherein the spacer is configured to impart an angular expansion to the spinal implant.

12. A spinal implant according to claim 1, wherein the at least two cage ends and the spacer are joined together utilizing an adhesive, the at least two cage ends comprising grooves or serrations configured to interlock with the spacer.

13. A spinal implant according to claim 11, wherein the at least two cage ends comprise horizontal grooves or serrations positioned at an angle from a horizontal on a side adjacent to or facing the spacer.

14. A spinal implant according to claim 12, wherein an angle to distract adjacent vertebrae from a horizontal can vary from about 0° to 12°.

15. A method for fusing two adjacent vertebrae comprising: placing an intervertebral cage having at least two ends into an intervertebral space adjacent vertebrae in need of fusion, the intervertebral cage configured to receive a spacer between the at least two ends, the spacer comprising a length that is smaller than a length of the at least two ends; placing the spacer into the intervertebral cage so as to increase distance between the adjacent vertebrae in need of fusion, wherein the intervertebral cage and the spacer are made entirely of allograft bone and the allograft bone is positively charged or negatively charged.

16. A method according to claim 15, wherein each end of the intervertebral cage comprises grooves or serrations configured to interlock with the spacer.

17. A method according to claim 15, wherein the intervertebral cage is placed using a cannula.

18. A method according to claim 15, wherein the spacer is in a shape of a dowel, a wedge, a cylinder, a prism, a cuboid, a cone or a pyramid.

19. A method according to claim 15, wherein the intervertebral cage and the spacer are used in a surgical procedure comprising a posterior lumbar interbody fusion, transforaminal lumbar interbody fusion, an anterior lumbar interbody fusion or a direct lateral interbody fusion.

20. A method of treating a spinal defect, the method comprising: preparing a disc space between adjacent vertebrae having the spinal defect to receive a spinal cage, the spinal cage comprising two ends opposite each other and a channel disposed between the two ends configured for receiving a spacer, the spacer comprising a length that is smaller than a length of the two ends; implanting the spinal cage into the disc space; inserting the spacer into the channel between the two ends of the spinal cage; and orienting the spacer for interlocking with the spinal cage so as to increase distance between the adjacent vertebrae, wherein the spinal cage and the spacer are made entirely of allograft bone and the allograft bone is positively charged or negatively charged.
